(19) United States
(12) Patent Application Publication
(10) Pub. No.: US 2024/0293619 A1
(43) Pub. Date: Sep. 5, 2024

(54) METHOD TO IMPROVE SAFETY MONITORING IN TYPE-1 DIABETIC PATIENTS BY DETECTING IN REAL-TIME FAILURES OF THE GLUCOSE

(71) Applicant: Dexcom, Inc., San Diego, CA (US)

(72) Inventors: Andrea Facchinetti, Padova (IT); Simone Del Favero, Padova (IT); Giovanni Sparacino, Padova (IT); Claudio Cobelli, Padova (IT)

(21) Appl. No.: 18/614,977

(22) Filed: Mar. 25, 2024

Related U.S. Application Data
(63) Continuation of application No. 15/067,104, filed on Mar. 10, 2016, now abandoned.
Application No. 15/067,104 is a continuation of application No. 13/785,384, filed on Mar. 5, 2013, now abandoned.
(60) Provisional application No. 61/606,542, filed on Mar. 5, 2012.

(51) Int. Cl. A61M 5/172; A61B 5/00; A61B 5/145; A61M 5/14; A61M 5/142; G16H 20/17; G16H 40/63; G16H 40/67; G16Z 99/00

(52) U.S. Cl. CPC A61M 5/1723; A61B 5/14532; A61B 5/4839; A61B 5/725; A61B 5/7282; A61B 5/7405; A61B 5/742; A61B 5/743; A61B 5/7455; A61B 5/746; A61M 5/14; A61M 5/14276; G16H 20/17; G16H 40/63; G16H 40/67; G16Z 99/00; A61M 2202/0468; A61M 2205/18

(57) ABSTRACT

A device for monitoring a diabetic patient includes continuous glucose monitoring system that is configured to generate glucose data indicative of the patient's actual glucose level. An continuous subcutaneous insulin infusion pump is configured to inject insulin into the patient and that is configured to generate insulin data regarding when and how much insulin has been injected into the patient. A processor, programmed with a discrete-time reiterative filter, calculates a predicted glucose level corresponding to a predicted glucose level currently expected to be sensed by the continuous glucose monitoring system, based on the insulin data and the glucose data over time and is also programed to generate an alert when the actual glucose level is different from the predicted glucose level by a predetermined amount. An alert generating device is coupled to the processor and is configured to generate an aesthetically-sensible event corresponding to the generation of the alert.

### CROSS-REFERENCE TO RELATED APPLICATION(S)

This application claims the benefit of U.S. Provisional Patent Application Ser. No. 61/606,542, filed Mar. 5, 2012, the entirety of which is hereby incorporated herein by reference.

### BACKGROUND OF THE INVENTION

### 1. Field of the Invention

The present invention relates to continuous glucose monitoring (CGM) sensors and insulin infusion pump devices, and, more specifically, to a method to detect in real-time failures of a system incorporating a CGM sensor and a continuous subcutaneous insulin infusion (CSII) pump.

### 2. Description of the Related Art

Diabetes is a disease that causes abnormal glycemic values due to the inability of the pancreas to produce insulin (Type-1 diabetes) or to the inefficiency of insulin secretion and action (Type-2 diabetes). Patients affected by diabetes need to monitor their glycemic level during all day in order to control it and take countermeasures to keep it inside the normal range of 70-180 mg/dL as much as possible.

In Type-1 patients, diabetes management is normally based on exogenous insulin infusions, whose scheduling and dosages are tuned on the basis of 3-4 finger-stick glucose measurements per day. Recently, new technologies have been developed in order to improve and facilitate diabetes therapy, such as: sensors for continuous glucose monitoring (CGM) devices, which are minimally invasive devices which return real-time glucose measures every several minutes, and pumps for continuous subcutaneous insulin infusion (CSII), which allow an effective and physiological delivery of insulin. However, in both daily-life/clinical use of sensor-augmented pumps and in artificial pancreas applications, a prompt detection of possible failures in either the CGM sensor or CSII pump is crucial for the safety of the patient.

Failures of the CGM sensor can result in: spikes, such as isolated CGM values which are significantly greater/lower than the expected glucose concentrations; transient losses of sensitivity of the CGM device, such as events due e.g. to a pressure applied to the sensor placed on the skin, which appear on glucose data as underestimations of the current glucose concentration for several consecutive samples; and drifts, such as persistent under/over estimations of glucose concentration, with error amplitude that increases with time.

The term “failures of the insulin infusion pump” usually but not solely refer to malfunctioning in the delivery of insulin, e.g., under/over delivering of insulin with respect to the nominal quantity programmed by the user/clinician, causing critical episodes of hyperglycemia and hypoglycemia. This means that when the pump is configured to deliver a nominal quantity of insulin X, while in actuality the insulin injected is Y, with Y>X in the case of over delivery and Y<X in the case of under delivery. Such failures can occur for several reasons, including: mechanical defects (which account for about 20% of the total number of failures); kinking; occlusion of the catheter; and simple pulling out of the catheter from the insertion site.

Therefore, there is a need for a system to alert appropriate personnel of failures in insulin infusion and glucose monitoring.

### SUMMARY OF THE INVENTION

The disadvantages of the prior art are overcome by the present invention which, in one aspect, is a device for monitoring a diabetic patient that includes a continuous glucose monitoring system, a continuous subcutaneous insulin infusion pump, a processor and an alert generating device. The continuous glucose monitoring system is configured to generate glucose data indicative of the patient's actual glucose level. The continuous subcutaneous insulin infusion pump is configured to inject insulin into the patient and that is configured to generate insulin data regarding when and how much insulin has been injected into the patient. The processor is in data communication with the continuous glucose monitoring system and the insulin pump. The processor is programmed with a discrete-time reiterative filter configured to calculate a predicted glucose level corresponding to a predicted glucose level currently expected to be sensed by the continuous glucose monitoring system, based on the insulin data and the glucose data over time. The processor is also programed to generate an alert when the actual glucose level is different from the predicted glucose level by a predetermined amount. The alert generating device is coupled to the processor and is configured to generate an aesthetically-sensible event corresponding to the generation of the alert.

In another aspect, the invention is an improvement to a glucose monitoring system for monitoring a diabetic patient that includes a continuous glucose monitoring system that is configured to generate glucose data indicative of the patient's actual glucose level and an insulin pump that is configured to inject insulin into the patient and that is configured to generate insulin data regarding when and how much insulin has been injected into the patient. The improvement includes a processor, in data communication with the continuous glucose monitoring system and the insulin pump, that is programmed with a failure detection module to calculate a predicted glucose level based on the insulin data and the glucose data over time and that is programed to generate an alert when the actual glucose level is different from the predicted glucose level by a predetermined amount.

In yet another aspect, the invention is a method of monitoring a diabetic patient in which glucose data is received from a continuous glucose monitoring system and is indicative of the patient's actual glucose level. Insulin data is received from an insulin pump. The insulin data is indicative of when and how much insulin has been injected into the patient. A predicted glucose level based on the glucose data and the insulin data is generated. The actual glucose level is compared to the predicted glucose level. An alert is generated when the actual glucose level is different from the predicted glucose level by a predetermined amount.

A method, which can be referred to as failure-detection module (FDM), receives in input glucose data measured by a continuous glucose monitoring (CGM) sensor (either subcutaneous or not), and information of insulin injected by an insulin pump, preferably a continuous subcutaneous insulin infusion (CSII) pump, and generates in output a failure alert when the value predicted by the method based on a model and the value measured by the glucose sensor are not consistent.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a block diagram of one embodiment of a failure alert system.

FIG. 2 is a block diagram describing the architecture of a failure detection module.

FIG. 3 is a block diagram showing one embodiment of a Kalman estimator.

FIGS. 4A-4C are a series of graphs demonstrating several examples of failures.

FIG. 5A-5C are a series of graphs showing three representative examples detection of CGM and CSII failures.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

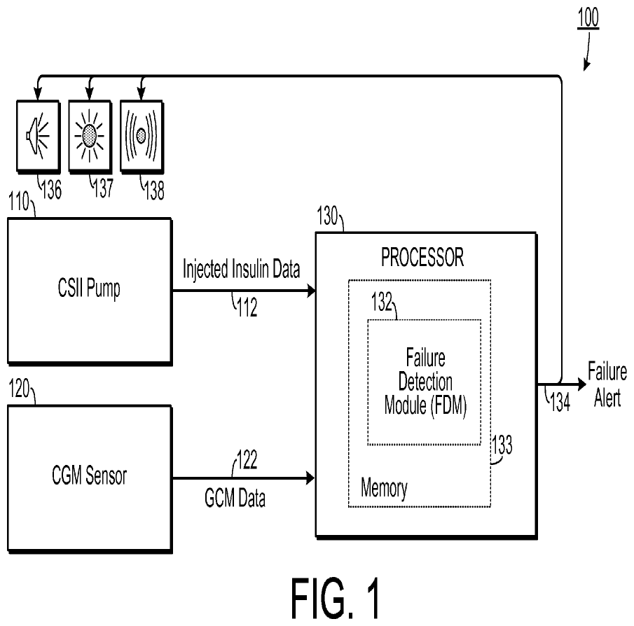

As shown in FIG. 1, one embodiment of device for monitoring a diabetic patient 100 includes a continuous subcutaneous insulin infusion (CSII) pump 110 having the ability to output insulin data 112 indicative of when and how much insulin has been pumped into the patient. A continuous glucose monitoring (CGM) sensor 120 is configured to sense the amount of glucose in the patient's blood stream at any given time and to generate glucose data 122 indicative of the amount of glucose detected. A processor 130 is programmed with a failure detection module (FDM) 132 that is stored in a tangible computer readable memory 133 (such as programmable logic array, a hard drive, a flash drive, or any other physical memory device, and combinations thereof) and that continuously calculates an amount of glucose that is predicted to be in the patient's bloodstream based on the insulin data 112 and the glucose data 122 over time. The processor also compares the predicted amount of glucose to the actual amount of glucose in the patient's bloodstream and generates a failure alert 134 when the difference is greater than a predetermined threshold. The failure alert 134 can be sensed in one or a combination of several ways. For example, it can be an audible alarm 136 a visual alarm 137 a vibrational alarm 138 or combinations thereof. The alarms can also be coupled to the insulin pump 110.

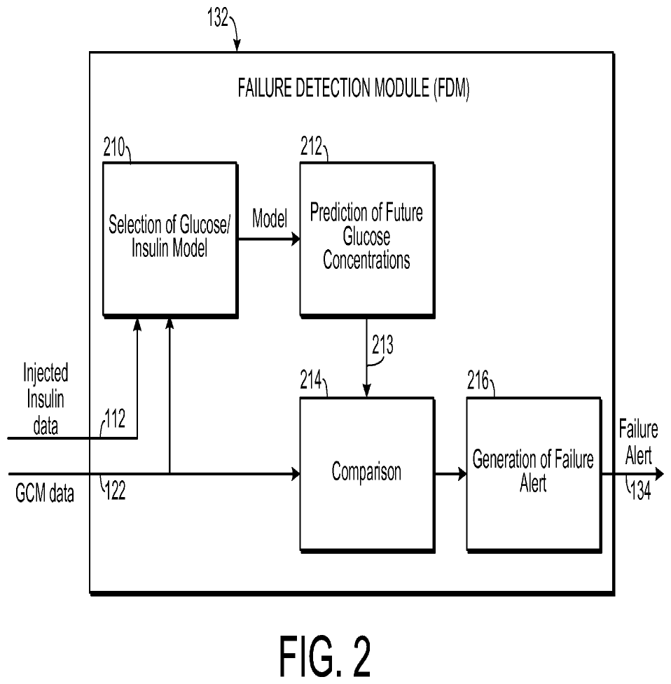

As shown in FIG. 2, one embodiment of the failure detection module 132 includes a routine 210 that selects a model that describes the relationship between glucose level data 122 measured by CGM sensor and insulin data 112 regarding insulin injected by the CSII pump. The selected model is input to a routine 212 that calculates a prediction of future glucose concentrations based on past glucose levels and past administration of glucose to the patient. The resulting prediction 213 of glucose level is input to a comparison routine 214 that compares the predicted glucose level 213 to the actual glucose level 122 received from the CGM. If the difference between the two is greater than a predetermined amount over a predetermined amount of time, then the FDM 132 generates a failure alert 216.

The models employed by the model selection routine 210 can be provided either externally to FDM, entirely derived within FDM or individualized based on patient's data. In one embodiment, the model selection routing 210 receives both the glucose level data 122 and the injected insulin data 112 to allow patient-specific individualization of the model of the glucose-insulin relationship.

Selection of the model that describes the relationship between glucose level measured by the CGM sensor and insulin injected by the CSII pump can involve several factors. When the option of model individualized to the patient is chosen, the model is identified from CGM and CSII data collected in the patient during a burn-in interval. In addition, different models, either physiological or input-output, can be used to describe different features of the system (low frequency components, high frequency components, etc.). In one embodiment of the invention, a discrete state-space model in the innovation the following form may be employed:

$$x ( t + 1 ) = Ax ( t ) + Bu ( t ) + Ke ( t ) \qquad ( 1 a )$$

$$y ( t ) = Cx ( t ) + Du ( t ) + e ( t ) \qquad ( 1 b )$$

In Eqs. (1a)−(1b), x(t) is the state vector at discrete time t, u(t) is the amount of insulin injected by the pump at the sampling time t, e(t) is the innovation process (with variance estimated from the data), and y(t) is the glucose level measured by the CGM sensor at time t. For instance, the identification of the model can be performed by resorting to a modified version of the numerical algorithms for subspace state space system identification (N4SID) approach, a numerical algorithm for subspace state identification designed to suitably handle with closed-loop systems such as the glucose-insulin model. Other possible models that can be employed in this step include black-box input-output models, such as autoregressive with exogenous inputs (ARX), autoregressive-moving average with exogenous inputs (ARMAX) or Box-Jenkins nonparametric models based on stable splines as specifically applied to diabetes, or neural networks. All these models allow the prediction of future glucose concentrations.

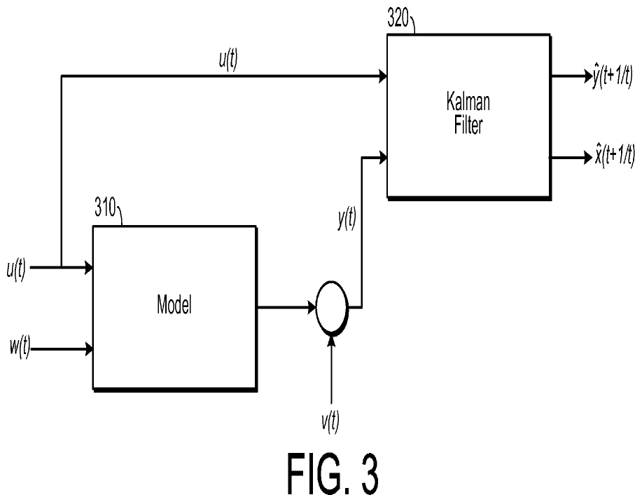

As shown in FIG. 3, the model 210 typically incudes a mathematical description of the glucose-insulin relationship expected in the patient. Input u(t) is the insulin injected by the pump, w(t) and v(t) are white noises, and y(t) is the glucose level measured by the CGM sensor. Outputs ŷ(t+1|t) and \hat{x}(t+1|t) are the one-step ahead predicted glucose level and predicted state-vector in the delayed form, respectively.

Based on the model uploaded or created, a discrete-time predictor is derived. This embodiment employs a discrete-time Kalman filter predictor. The Kalman filter inputs are glucose concentration y(t) and insulin infusion u(t), and the output is the one-step ahead prediction of the glucose concentration ŷ(t|t−1). Since the model selection routine 210 (shown in FIG. 2) gives a model in innovation form, the Kalman filter prediction can be easily obtained by computing at each time instant the innovation

$$e ( t ) = y ( t ) - \hat{y} ( t \vert t - 1 ) \qquad ( 2 )$$

and plugging e(t) in Eqs. (1a) and (1b):

$$\hat{x} ( t + 1 \vert t ) = A \hat{x} ( t \vert t - 1 ) + Bu ( t ) + Ke ( t ) \qquad ( 3 a )$$

$$\hat{y} ( t + 1 \vert t ) = C \hat{x} ( t \vert t - 1 ) + Du ( t ) \qquad ( 3 b )$$

Starting from the system identified using subspace identification procedure (as in the model selection routine 210), the Kalman filter 320 may be derived, for example, by using the “Kalman” function of Matlab®). The derivation is performed in the delayed form. This means that ŷ(t|t−1) is estimated using glucose sensing data till time t−1, while insulin information is used till time t. In practice, the system predicts how CGM is going to change given the next (known) insulin infusion.

Returning to FIG. 2, the predicted glucose level 213 given by ŷ(t|t−1) obtained is compared with glucose concentrations 122 measured by the CGM sensor in c comparison routine 214. For sake of simplicity, and without any loss of generality, hereafter we consider only a one-step ahead prediction embodiment. However, predictions of two-steps ahead, three-steps ahead, . . . , k-steps ahead of glucose level can be performed by re-iterating the prediction model while using new values of infused insulin in the prediction model of Equations. (3a)-(3b). The comparison 214 can be performed by employing various statistical tools. In one embodiment, the comparison consists in evaluating whether y(t) overcomes a confidence interval given by (ŷ(t|t−1)−mSD, ŷ(t|t−1)+mSD), where SD

$$SD = \sqrt{Var [ e ]} \qquad ( 4 )$$

is the standard deviation of the estimated value, Var[e] is the variance of the innovation process estimated from the data by the subspace identification procedure, and m is a suitable positive integer (e.g. m=2). The equality in Eq. (4) is possible since the identified model is innovation form, so that SD is simply the square root of the variance of the innovation.

If the result of the comparison 214 indicates the presence of an inconsistency, then a failure alert is generated 216. In one embodiment, every time y(t) overcomes the confidence interval (ŷ(t|t−1)−mSD, ŷ(t|t−1)+mSD), a failure alert is generated. The failure alert can be given in form of sound, vibration, visual information (e.g., through the flashing of a light or the appearance of a visual alert icon on a video monitor screen), or combinations of such alerts.

### ASSESSMENT OF THE INVENTION

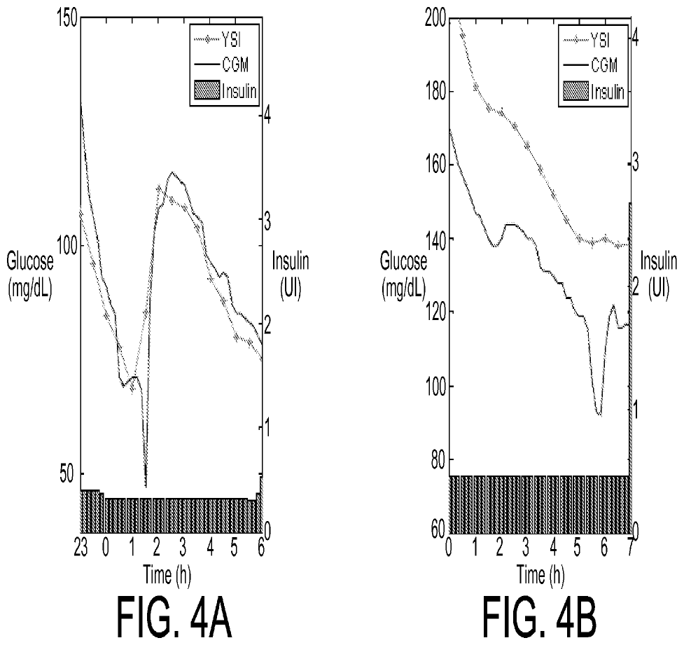

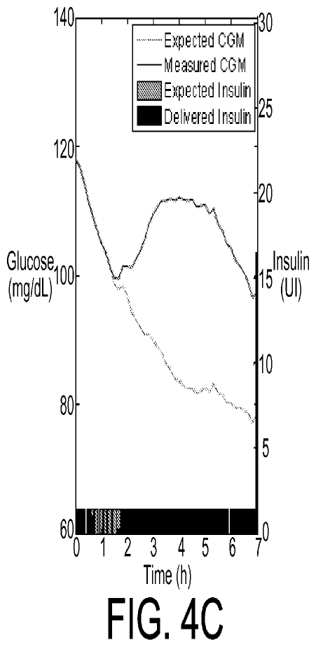

Several examples of nighttime failures are shown in FIGS. 4A-4C. FIG. 4A shows a spike failure on CGM data (black line) at time 01 h30 m; FIG. 4B shows a transient loss of sensitivity failure in CGM data from 05 h10 m to 06 h00 m; and FIG. 4C shows a CSII pump failure at 00 h40 m, whose effect if visible starting from 01 h50 m. The first two examples (shown in FIGS. 4A-4B) refer to nighttime monitoring of two Type-1 diabetic patients whose data has been collected in experiments documented in clinical observation, while the third example (shown in FIG. 4C) is produced using a Type-1 diabetic simulator approved by the Food and Drug Administration. From a clinical point of view, failures occurring during daytime may be less critical because the patient is awake and can promptly detect and fix them. The nighttime scenario is more dangerous because the patient is asleep and often cannot take timely countermeasures.

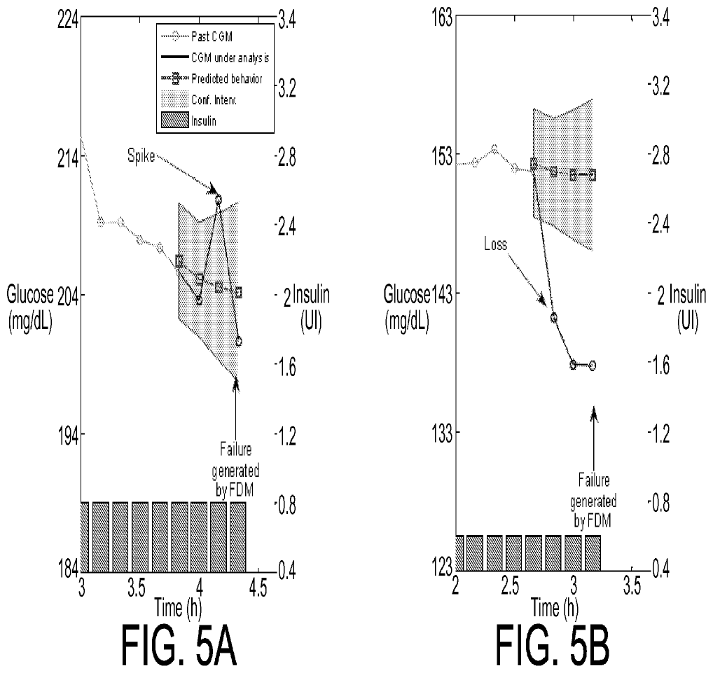

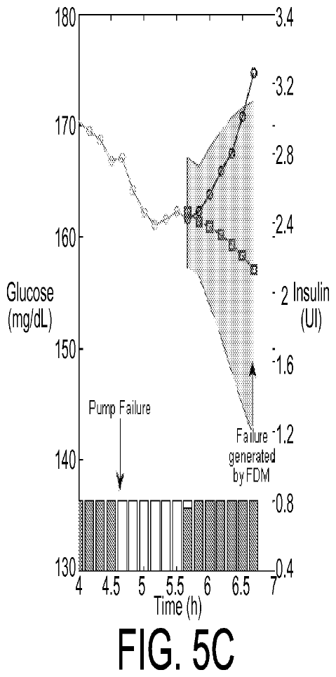

FIGS. 5A-5C demonstrate how FDM works in real time in these three possible failure scenarios (spike, transient loss of sensitivity, and pump failures). CGM data are represented with circles (the line between circles is a simple linear interpolation used to assure a better visualization of the trace). FDM prediction and its confidence interval are represented by black squares and grey area, respectively.

The scenario shown in FIG. 5A demonstrates a failure alert being generated at time 04 h20 m. A spurious spike at time 04 h10 m was present. FDM prediction calculated at time 04 h20 m, i.e. at the current time instant, using CGM data through 03 h50 m and injected insulin data through 04 h20 m. FDM compares the three CGM values with the corresponding predictions, and detects that the value at 04 h10 m overcomes the confidence interval and that the next value jumps back inside it. Therefore, FDM generates a failure alert at time 04 h20 m.

The scenario shown in FIG. 5B demonstrates a failure alert being generated at time 03 h10 m. A transient loss of sensitivity was present at time 02 h50 m. FDM compared the three CGM values with the prediction. All three samples fall outside of it and thus FDM generates a failure alert.

The scenario shown in FIG. 5C demonstrates a failure alert being generated at time 06 h40 m. The pump failure consisted in a stop in the insulin delivery starting at 04 h40 m. The failure lasted for 1 hour. Because of slow modifications in glucose concentration profile due to insulin action/absorption, a short PH (=30 min) will not be a suitable solution to catch such a failure. This explains why, here, a longer PH is selected, in this case 60 min. At time 06 h40 m, a failure alert is generated.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A device for monitoring a diabetic patient, comprising:
(a) a continuous glucose monitoring system that is configured to generate glucose data indicative of the patient's actual glucose level;
(b) a continuous subcutaneous insulin infusion pump that is configured to inject insulin into the patient and that is configured to generate insulin data regarding when and how much insulin has been injected into the patient;
(c) a processor, in data communication with the continuous glucose monitoring system and the insulin pump, that is programmed with a discrete-time reiterative filter configured to calculate a predicted glucose level corresponding to a predicted glucose level currently expected to be sensed by the continuous glucose monitoring system, based on the insulin data and the glucose data over time and that is programed to generate an alert when the actual glucose level is different from the predicted glucose level by a predetermined amount; and
(d) an alert generating device coupled to the processor and configured to generate an aesthetically-sensible event corresponding to the generation of the alert.

2. The device of claim 1, wherein the discrete-time reiterative filter includes a Kalman filter predictor that is configured to calculate the predicted glucose level.

3. The device of claim 1, wherein the alert generating device comprises a sound generating device.

4. The device of claim 1, wherein the alert generating device comprises a light generating device.

5. The device of claim 1, wherein the alert generating device comprises a vibration generating device.

6. In a glucose monitoring system for monitoring a diabetic patient that includes a continuous glucose monitoring system that is configured to generate glucose data indicative of the patient's actual glucose level and an insulin pump that is configured to inject insulin into the patient and that is configured to generate insulin data regarding when and how much insulin has been injected into the patient, the improvement comprising:
a processor, in data communication with the continuous glucose monitoring system and the insulin pump, that is programmed with a failure detection module to calculate a predicted glucose level based on the insulin data and the glucose data over time and that is programed to generate an alert when the actual glucose level is different from the predicted glucose level by a predetermined amount.

7. The glucose monitoring system of claim 6, wherein the insulin pump comprises a continuous subcutaneous insulin infusion pump.

8. The glucose monitoring system of claim 6, wherein the failure prediction module includes a discrete-time Kalman filter predictor that is configured to calculate the predicted glucose level.

9. The glucose monitoring system of claim 6, wherein the alert comprises an audible alarm.

10. The glucose monitoring system of claim 6, wherein the alert comprises a visual notification.

11. The glucose monitoring system of claim 6, wherein the alert comprises a vibration.

12. A method of monitoring a diabetic patient, employing a processor coupled to a tangible computer-readable memory, comprising the steps of:
(a) receiving glucose data, from a continuous glucose monitoring system, indicative of the patient's actual glucose level;
(b) receiving insulin data, from an insulin pump, indicative of when and how much insulin has been injected into the patient.
(c) generating a predicted glucose level based on the glucose data and the insulin data;
(d) comparing the actual glucose level to the predicted glucose level; and
(e) generating an alert when the actual glucose level is different from the predicted glucose level by a predetermined amount.

13. The method of claim 12, wherein the insulin pump comprises a continuous subcutaneous insulin infusion pump.

14. The method of claim 12, wherein the step of generating a predicted glucose level employs a discrete-time Kalman filter predictor that is configured to calculate the predicted glucose level.

15. The method of claim 12, wherein the alert comprises an audible alarm.

16. The method of claim 12, wherein the alert comprises a visual notification.

17. The method of claim 12, wherein the alert comprises a vibration.
